(19) United States
(12) Patent Application Publication
(10) Pub. No.: US 2026/0242315 A1
(43) Pub. Date: Aug. 20, 2026

(54) Process for Producing Guerbet Alcohols from Fusel Alcohols

(71) Applicant: Terra Mater BV, Gent (BE)

(72) Inventor: Kapil Shyam Lokare, Kampenhout (BE)

(21) Appl. No.: 19/161,699

(22) PCT Filed: Mar. 1, 2024
(86) PCT No.: PCT/EP2024/055345

§ 371 Date: Sep. 2, 2025

(30) Foreign Application Priority Data
Mar. 2, 2023 (EP) 23159582.8

(51) Int. Cl. C07C 29/60; B01J 23/06; B01J 31/24

(52) U.S. Cl. CPC C07C 29/60; B01J 23/06; B01J 31/2404; B01J 2231/342; B01J 2531/004; B01J 2531/821; B01J 2531/842

(57) ABSTRACT

The invention relates to a process for dimerising a C₅₋₂₀ alcohol to a C₁₀₋₄₀ alcohol, the process comprising the steps of: contacting the C₅₋₂₀ alcohol with a catalytic mixture comprising: —a base; —a catalyst comprising a (L)ₙM(OH)ₙ(H₂O)ₘ type complex; wherein M is a metal selected from the group comprising: Fe, Ru, Os, Zn, and Ni; and, wherein L is a ligand; —a Zn additive; and, —a solvent; and, —dimerising the C₅₋₂₀ alcohol in the catalytic mixture to form a C₁₀₋₄₀ alcohol.

### FIELD OF THE INVENTION

The invention relates to a process for the preparation of Guerbet alcohols from fusel alcohols.

### BACKGROUND OF THE INVENTION

Fusel oils are produced during ethanol production as by-products of the yeast metabolism. These alcohols are higher boiling and volatile compared to ethanol, and therefore, a distillation step enables their separation from fermented mash. These are a mixture of primarily alcohols, including active amyl alcohol (2-methyl-1-butanol), isoamyl alcohol (3-methyl-1-butanol), iso-butyl alcohol, 1-propanol and, in lesser amounts, n-amyl alcohol, n-butyl alcohol, and methionol. Less volatile alcohols are also present in the mash; and are poorly extracted by distillation, including phenethyl alcohol and tyrosol. Some of these alcohols are aromatic and are associated with strong tastes and pungent odours. Used sparingly, fusel alcohols and esters can contribute positively to food and beverage flavours, but at higher concentrations, they are associated with unpleasant flavours and “hangover” symptoms.

3-methyl-1-butanol (isoamyl alcohol) is the main constituent of the fusel oil. Alternatively, 3-methyl-1-butanol can not only be obtained from fusel oil, but, e.g., also by hydroformylation followed by hydrogenation of butene isomer mixture to give a mixture of alcohols consisting of 3-methyl-1-butanol, 2-methyl-1-butanol, and 1-pentanol.

Guerbet alcohols have been known since 1909. This chemistry has made possible the synthesis of a regiospecific, β-branched hydrophobe, which introduces branching with a very high degree of regiospecificity. The ability to capitalise upon this reaction sequence and develop derivatives has resulted in the preparation of many materials that find use in applications where liquidity and lubrication are important, such as in metal lubrication, plastic mould release, paper processing, synthetic lubricants (synlube), and personal-care products. The chemistry results in a unique class of materials.

The reaction sequence for producing Guerbet alcohols is related to the aldol reaction and occurs at high temperatures under catalytic conditions. The overall reaction can be represented by FIG. 1. The product is a β-branched primary alcohol with twice the molecular weight of the reactant alcohol minus a mole of water. The reaction proceeds by several sequential steps: (i) oxidation of alcohol to aldehyde, (ii) aldol condensation after proton extraction, (iii) dehydration of the aldol product, and (iv) hydrogenation of the allylic aldehyde.

Many catalysts have been described in the literature as effective for the preparation of Guerbet alcohols including, nickel, lead salts, and oxides of copper, lead, zinc, chromium, molybdenum, tungsten, and manganese. Later developments include palladium compounds and silver compounds, and the use of an expensive iridium catalyst with the use of a diene, 1,7-octadiene.

A first major disadvantage is the formation of several alcohols of higher molecular weight, C₃ₓ, C₄ₓ and so on, where ‘x’ denotes the initial number of carbon atoms. Ideally, the conversion should yield selectively C₂ₓ with conversions of up to 50% for economic viability.

A second disadvantage is the Cannizzaro reaction, which is a major side reaction in such processes and is described as the disproportionation of two molecules of an aldehyde, brought about by the action of sodium or potassium hydroxide to yield the corresponding alcohol and acid.

The extent to which these side reactions take place depends on the case and the nature of the starting alcohol, catalyst, and the reaction conditions.
1. The reaction can take place without a catalyst, but it is strongly catalysed in the presence of hydrogen transfer catalysts.
2. At low temperatures (130° C. to 140° C.), the oxidation process (i.e., formation of the aldehyde) is the rate-limiting step.
3. At somewhat higher temperatures (160° C. to 180° C.), the rate limiting step is the aldol condensation.
4. At even higher temperatures (220° C. to 350° C.), other degradative reactions occur and may become dominant.

Most processes described in patent literature operate at 220 to 300° C. Such processes work when the raw materials are primary alcohols of natural origin C₁₀₋₁₂ onwards, with even-numbered, straight carbon chains. However, in the production of short-chain Guerbet alcohols having less than 16 carbon atoms starting from C₆₋₈ alcohols, vapour pressures of more than 20 bar over a residence time of over 40 hours are achieved in this temperature range, which place very high technical demands on the production systems and are therefore difficult to implement. Such harsh conditions also lead to several side product streams that need to be subsequently removed by rigorous distillation techniques.

Therefore, thus far, oxo-alcohols with lower carbon numbers (C₄, C₅, C₆) have found limited use in Guerbet reactions as both reaction rate and conversions are lower, limiting their scope on an industrial scale.

Thus, produced on an industrial scale, Guerbet fatty alcohols can be subjected to a series of post-reaction steps that (i) remove unreacted alcohol (vacuum stripping), (ii) remove unsaturation (hydrogenation), (iii) remove Cannizzaro soap (filtration), and/or (iv) remove colour/odour bodies. These operations add to the cost of production.

The commercial C₁₀ alcohol that is available from fossil routes is 2-propyl heptanol. A first step comprises the preparation of aldehydes via a mixture of butenes and syn-gas over rhodium catalysts in a process known as the LP Oxo Process. This gives several products, typically occurring in four different structural isomers as 1-pentanal, 2-methylbutanal, 3-methylbutanal, and 2,2-dimethylpropanal or pivalaldehyde. These pentanals may be used as a mixture, for example for the production of fragrances, or in the form of the derivatization products thereof, such as pentanols, pentanoic acids, or pentylamines. The pentanals can be processed in pure isomeric form or in the form of an isomer mixture. A second step comprises converting the mixture of aldehydes with KOH to give corresponding aldol condensation products, i.e., decanals, and subsequent hydrogenation to give decanol isomeric mixture commercially sold as 2-propyl heptanol (which consists of 85% 2-propyl heptanol and 15% 2-isopropyl-5-methyl-1-hexanol). These have boiling points within at 5° C. of each other, are thus never separated, and therefore never available in pure form. If the aldehydes are hydrogenated before aldol condensation one would get several isomeric pentanols as 1-pentanol, 2-pentanol, 3-pentanol, 2-methyl-1-butanol, 3-methyl-1-butanol, 2-methyl-2-butanol, 2-methyl-3-butanol, or 2,2-dimethylpropanol. To obtain access to these individual alcohols requires tedious distillations as these have boiling points within 3-5° C. of each other. The only way forward would be to purify these individual aldehydes and then hydrogenate them individually to get pure alcohols. However, when heating these aldehydes under distillation conditions, they have to tendency to decompose rapidly.

Accordingly, it is an object of the present invention to provide a process that overcomes one or more of the above issues.

More specifically, it is an object of the invention to provide an improved process for the dimerisation of alcohols, particularly the conversion of isoamyl alcohol (3-methyl-1-butanol), to a C₁₀ alcohol such as 2-isopropyl-5-methyl-1-hexanol. It is also an object of the invention to provide a process that proceeds with good conversions, preferably with yields >60% per pass.

It is also an object of the invention to provide a process that has little to no formation of by-products. It is also an object of the invention to provide a process that can operate at similar or lower reaction temperatures than those commonly used, preferably less than 220° C. to 300° C. It is also an object of the invention to provide a process that can operate at similar or lower reaction pressures than those commonly used, preferably less than 20 bar. It is also an object of the invention to provide a process that can provide similar or shorter residence times than those commonly used, preferably less than 24 hours. It is also an object of the invention to provide a process that is resource efficient, i.e., catalyst cost and losses should be kept within technically acceptable limits. It is also an object of the invention to provide a process that avoids the use of an expensive catalyst system. It is also an object of the invention to provide a process wherein the reaction mixture may be prepared in air.

### SUMMARY OF THE INVENTION

The present inventors have now found that one or more of the objects above can be attained by using the process as presently claimed, and (preferred) embodiments thereof.

An advantage of the present invention is that the Guerbet reaction proceeds with good conversions (>60% per pass).

A further advantage of the present invention is that by-product formation is negligible. Here, intermediates and structural isomers of the Guerbet reaction are considered as conventional by-products and can be limited to preferably less than 5.0 wt %, more preferably less than 2.0 wt % and most preferably less than 1.0 wt % as identified via gas chromatography. Typical by-products would be the intermediates in the Guerbet reaction of isoamyl alcohol, i.e., isovaleraldehyde (3-methylbutanal), 2-isopropyl-5-methyl-2-hexenal; and 2-isopropyl-5-methylhexanal. In embodiments of the invention, no isovaleraldehyde was observed at all, while 2-isopropyl-5-methyl-2-hexenal and 2-isopropyl-5-methylhexanal were only formed in limited amounts.

A further advantage of the present invention is that reaction temperatures, pressures, and/or residence times are lower compared to the prior art. This renders the process more ecologically and energetically friendly.

A further advantage of the present invention is that it uses an inexpensive catalyst system and that the process is resource efficient. A further advantage of the present invention is that this type of catalyst is air stable. This allows the reaction mixture to be prepared in air.

Another advantage of this procedure is that it eliminates the need for intermediate distillation of thermally decomposable aldehydes or other intermediates with only a minor difference in boiling point obtained via the Guerbet reaction or other alcohol dimerization processes that are well-known to a person skilled in the art. Embodiments of the present invention circumvent typical issues with the commercial fossil route starting from 2-propyl heptanol; by starting with isoamyl alcohol that is obtained relatively cleanly as a side stream from bio-ethanol production and thus avoids multiple purification steps as outlined above to give 2-isopropyl-5-methyl-1-hexanol which can be used in appropriate chemical industry.

According to a first aspect, the present invention relates to a process for dimerising a C₅₋₂₀ alcohol to a C₁₀₋₄₀ alcohol. The process preferably comprises the steps of:
contacting the C₅₋₂₀ alcohol with a catalytic mixture; and,
dimerising the C₅₋₂₀ alcohol in the catalytic mixture to form a C₁₀₋₄₀ alcohol.

The catalytic mixture preferably comprises:
a base;
a catalyst comprising a (L)ₙM(OH)ₙ(H₂O)ₘ type complex;
   wherein M is a metal selected from the group comprising: Fe, Ru, Os, Zn, Ni;
   and, wherein L is a ligand;
a Zn additive; and,
a solvent.

In some preferred embodiments, the C₅₋₂₀ alcohol is a C₅ alcohol. Most preferably, the C₅₋₂₀ alcohol is isoamyl alcohol. In some preferred embodiments, the C₁₀₋₄₀ alcohol is a C₁₀ alcohol. Most preferably, the C₁₀₋₄₀ alcohol is 2-isopropyl-5-methyl-1-hexanol.

In some preferred embodiments, the metal M is Fe or Ru, preferably Fe. In some preferred embodiments, the catalyst comprises an iron dichloride hydrate or solvate salt, or a ruthenium trichloride hydrate or solvate salt; preferably an iron dichloride hydrate or solvate salt.

In some preferred embodiments, the ligand L is selected from the group comprising: a halide, a hydride, an alkoxide, an aryloxide, an amide, an acetate, an acetylacetonate, an alkyl, an aryl, CO, NO, phosphine, pyridine, an alkene, an alkyne, N-heterocyclic carbene, cyclopentadiene, a mono-dentate phosphine of formula PR¹R²R³, a bidentate phosphine such as R¹R²P—PR¹R², a methylene linked R¹R²P—CH₂—PR¹R², R¹R²P—(CH₂)ₙ—PR¹R², or R¹R²P—(CH₂)ₙ—NR⁴—(CH₂)ₙ—PR¹R²; wherein each R¹, R², R³ and/or R⁴ is independently selected from alkyl or aryl, with optional variable functionality such as sulfonate, halide, and wherein R¹, R², R³, and R⁴ can be identical or different; preferably wherein R¹, R², R³, and R⁴ are all phenyl.

In some preferred embodiments, the ligand L is selected from the group comprising: a mono-dentate phosphine of formula PR¹R²R³, a bidentate phosphine such as R¹R²P—PR¹R², a methylene linked R¹R²P—CH₂—PR¹R², R¹R²P—(CH₂)ₙ—PR¹R², or R¹R²P—(CH₂)ₙ—NR⁴—(CH₂)ₙ—PR¹R²; wherein each R¹, R², R³ and/or R⁴ is independently selected from alkyl or aryl, with optional variable functionality such as sulfonate, halide, and wherein R¹, R², R³, and R⁴ can be identical or different; preferably wherein R¹, R², R³, and R⁴ are all phenyl.

In some preferred embodiments, the base is selected from the group comprising: potassium hydroxide, sodium hydroxide, potassium isoamylate, sodium isoamylate, potassium t-butoxide, sodium t-butoxide, or combinations thereof. In some preferred embodiments, the base comprises potassium hydroxide.

In some preferred embodiments, the solvent is selected from the group comprising: poly(ethylene glycol) (PEG) monoalkyl ethers, aliphatics, aromatics, aromatics with aliphatic substitutions, higher boiling alcohols, xylenes, cycloalkanes, substituted cycloalkanes, naphthenes, indenes, fluorene, biphenyls, Petrosolv 200-300, Petroflux ND, Petrosolv 250-450, SOLGAD 150, SOLGAD 200, SOLGAD 200 ULN, SOLGAD 150 ULN, sulfolane, dimethyl sulfoxide, dimethyl formamide, N-methyl pyrrolidone, N, N-dimethyl acetamide, 1,4-dioxane, anisole, propylene carbonate, benzyl alcohol, N-methylpyrrolidone, N-ethylpyrrolidone, N-cyclohexylpyrrolidone, N-octylpyrrolidone, ethyllactate, butyllactate, morpholine, glycerin, glycerin-mono-tert-butyl-ether, glycerin-di-tert-butyl-ether, glycerin-tri-tert-butyl-ether, acetonitrile, propionitrile, di-arylether, alkyl aryl ethers, ionic liquids, dicarboxylic/tricarboxylic ester-based plasticisers, bis(2-ethylhexyl) phthalate, diisononyl phthalate, bis(n-butyl) phthalate, butyl benzyl phthalate, diisodecyl phthalate, di-n-octyl phthalate, diisooctyl phthalate, diethyl phthalate, diisobutyl phthalate, di-n-hexyl phthalate, trimellitates, trimethyl trimellitate, tri-(2-ethylhexyl) trimellitate, tri-(n-octyl,n-decyl) trimellitate, tri-(heptyl, nonyl) trimellitate, n-octyl trimellitate, bis(2-ethylhexyl) adipate, dimethyl adipate, monomethyl adipate, dioctyl adipate, sebacate-based plasticisers, dibutyl sebacate, maleates, dibutyl maleate, diisobutyl maleate, benzoates, epoxidised vegetable oils, sulfonamides, N-ethyl toluene sulfonamide (ortho and para isomers), N-(2-hydroxypropyl)benzene sulfonamide, N-(n-butyl)benzene sulfonamide, organophosphates, tricresyl phosphate, tributyl phosphate, glycols, polyethers, triethylene glycol dihexanoate, tetraethylene glycol diheptanoate, polybutene, acetylated monoglycerides, alkyl citrates, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trihexyl o-butyryl citrate, trimethyl citrate, alkyl sulphonic acid phenyl ester, vinyl chloride copolymers, 1,2-cyclohexane dicarboxylic acid diisononyl ester, biphenyl, triphenylmethane, polyaromatic hydrocarbons, tetralin, naphthalene, tetrahydro anthracene, anthracene, hexahydro pyrene, trihydropyrene, pyrene, phenanthrene, chrysene, paraffins, paraffinic intermediates, light naphtha, mid-naphtha, light gas oil, mid gas oil, heavy gas oil, light vacuum gas oil, mid-vacuum gas oil, heavy vacuum gas oil, vacuum residue, linear alkyl benzenes, vegetable oils, fatty acid methyl esters, animal fats/tallow, optionally substituted derivatives thereof, and combinations thereof. In some preferred embodiments, the solvent comprises xylenes.

In some preferred embodiments, the step of dimerising the C₅₋₂₀ alcohol in the catalytic mixture to form a C₁₀₋₄₀ alcohol, is performed at a temperature of at least 120° C. and at most 200° C. In some preferred embodiments, the step of dimerising the C₅₋₂₀ alcohol in the catalytic mixture to form a C₁₀₋₄₀ alcohol, is performed at a pressure of at least 0.01 bar and at most 10 bar. In some preferred embodiments, the step of dimerising the C₅₋₂₀ alcohol in the catalytic mixture to form a C₁₀₋₄₀ alcohol, is performed during a residence time of at least 30 minutes to at most 6 hours.

In some preferred embodiments, the step of dimerising the C₅₋₂₀ alcohol in the catalytic mixture to form a C₁₀₋₄₀ alcohol, is performed in a continuous flow system, preferably selected from the group comprising: a CSTR, a tubular reactor, or a microchannel reactor apparatus.

According to a second aspect, the present invention relates to use of a catalyst as described herein, in the dimerisation of a C₅₋₂₀ alcohol to a C₁₀₋₄₀ alcohol.

The independent and dependent claims set out particular and preferred features of the invention. Features from the dependent claims may be combined with features of the independent or other dependent claims as appropriate. The above and other characteristics, features and advantages of the present invention will become apparent from the following detailed description.

### BRIEF DESCRIPTION OF THE FIGURES

FIG. 1 illustrates the general Guerbet reaction.

FIG. 2 illustrates the specific formation of 2-isopropyl-5-methyl-1-hexanol from isoamyl alcohol.

FIG. 3 illustrates the temperature versus pressure co-relation for the production of 2-isopropyl-5-methyl-1-hexanol from isoamyl alcohol, and illustrates the difference between Fe and Ru, illustrating how iron operates at a much lower pressure than ruthenium, and is thus preferable.

FIG. 4 illustrates a schematic representation of a gas chromatograph of the crude phase conversion of isoamyl alcohol.

FIG. 5 illustrates the commercial production of 2-isopropyl-5-methyl-1-hexanol from isoamyl alcohol.

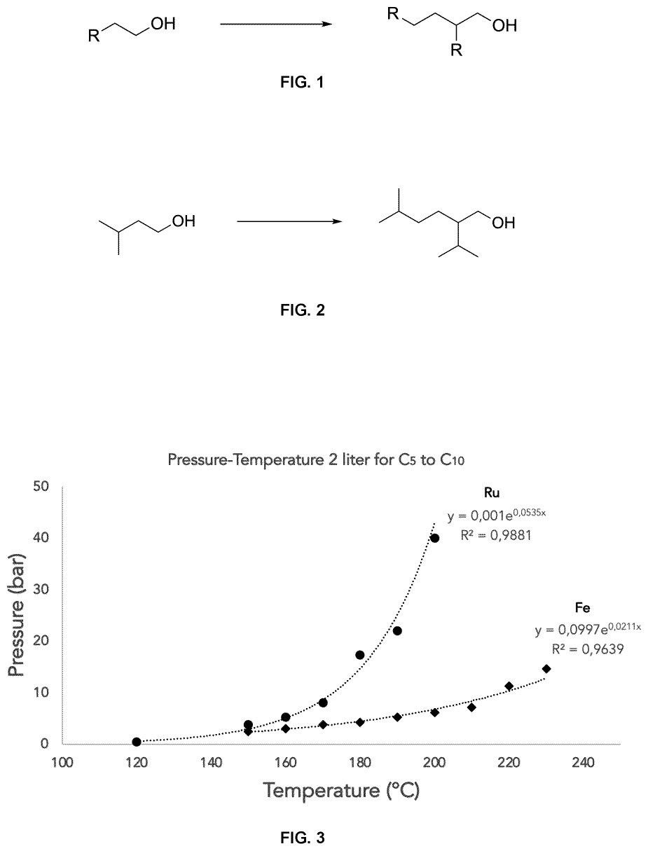

### DETAILED DESCRIPTION OF THE INVENTION

When describing the invention, the terms used are to be construed in accordance with the following definitions, unless a context dictates otherwise.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art.

The terms “comprising”, “comprises” and “comprised of” as used herein are synonymous with “including”, “includes” or “containing”, “contains”, and are inclusive or open-ended and do not exclude additional, non-recited members, elements, or method steps. It will be appreciated that the terms “comprising”, “comprises” and “comprised of” as used herein comprise the terms “consisting of”, “consists” and “consists of”.

As used in the specification and the appended claims, the singular forms “a”, “an,” and “the” include plural referents unless the context clearly dictates otherwise. By way of example, “a step” means one step or more than one step.

Unless defined otherwise, all technical and scientific terms used herein have the same meaning as is commonly understood by one of skill in the art. All publications referenced herein are incorporated by reference thereto.

The recitation of numerical ranges by endpoints includes all integer numbers and, where appropriate, fractions subsumed within that range (e.g., 1 to 5 can include 1, 2, 3, 4 when referring to, for example, a number of elements, and can also include 1.5, 2, 2.75 and 3.80, when referring to, for example, measurements). The recitation of endpoints also includes the end point values themselves (e.g., from 1.0 to 5.0 includes both 1.0 and 5.0). Any numerical range recited herein is intended to include all sub-ranges subsumed therein.

The term “about” as used herein when referring to a measurable value such as a parameter, an amount, a temporal duration, and the like, is meant to encompass variations of +/−10% or less, preferably +/−5% or less, more preferably +/−1% or less, of and from the specified value, insofar such variations are appropriate to perform in the disclosed invention. It is to be understood that the value to which the modifier “about” refers is itself also specifically, and preferably, disclosed.

When describing the present invention, the terms used are to be construed in accordance with the following definitions, unless a context dictates otherwise.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e., a compound that is sufficiently robust to survive isolation to a useful degree of purity from a reaction mixture.

The term “alkyl” as a group or part of a group, refers to a hydrocarbyl group of formula CₙH₂ₙ₊₁ wherein n is a number greater than or equal to 1. Alkyl groups may be linear or branched and may be substituted as indicated herein. Generally, alkyl groups of this invention comprise from 1 to 20 carbon atoms, preferably from 1 to 18 carbon atoms, preferably from 1 to 12 carbon atoms, preferably from 1 to 10 carbon atoms, preferably from 1 to 6 carbon atoms, more preferably 1, 2, 3, 4, 5, 6 carbon atoms. When a subscript is used herein following a carbon atom, the subscript refers to the number of carbon atoms that the named group may contain. For example, the term “C₁₋₂₀alkyl”, as a group or part of a group, refers to a hydrocarbyl group of formula —CₙH₂ₙ₊₁ wherein n is a number ranging from 1 to 20. Thus, for example, C₁₋₂₀alkyl groups include all linear, or branched alkyl groups having 1 to 20 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers, undecyl and its isomers, dodecyl and its isomers, tridecyl and its isomers, tetradecyl and its isomers, pentadecyl and its isomers, hexadecyl and its isomers, heptadecyl and its isomers, octadecyl and its isomers, and the like. For example, C₁₋₁₀alkyl includes all linear, or branched alkyl groups having 1 to 10 carbon atoms, and thus includes for example methyl, ethyl, n-propyl, i-propyl, 2-methyl-ethyl, butyl, and its isomers (e.g. n-butyl, i-butyl and t-butyl); pentyl and its isomers, hexyl and its isomers, heptyl and its isomers, octyl and its isomers, nonyl and its isomers, decyl and its isomers and the like. For example, C₁₆alkyl includes all linear, or branched alkyl groups having 1 to 6 carbon atoms, and thus includes for example methyl, ethyl, n-propyl, i-propyl, 2-methyl-ethyl, butyl, and its isomers (e.g. n-butyl, i-butyl, and t-butyl); pentyl and its isomers, hexyl and its isomers. When the suffix “ene” is used in conjunction with an alkyl group, i.e. “alkylene”, this is intended to mean the alkyl group as defined herein having two single bonds as points of attachment to other groups. Alkylene groups may be linear or branched and may be substituted as indicated herein. Non-limiting examples of alkylene groups include methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and its chain isomers, hexylene and its chain isomers.

The term “halo” or “halogen” or “halide”, as a group or part of a group, is generic for fluoro, chloro, bromo or iodo. The term “haloalkyl” as a group or part of a group, refers to an alkyl group having the meaning as defined above wherein one, two, or three hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl and the like.

The term “aryl”, as a group or part of a group, refers to a polyunsaturated, aromatic hydrocarbyl group having a single ring (i.e. phenyl) or multiple aromatic rings fused together (e.g. naphthyl), or linked covalently, typically containing 5 to 30 atoms, wherein at least one ring is aromatic. The aromatic ring may optionally include one to two additional rings (either cycloalkyl, heterocyclyl or heteroaryl) fused thereto. Examples of suitable aryl include C₆₋₃₀aryl, or C₆₋₁₈aryl, or C₆₋₁₂aryl, or C₆₋₁₀aryl, or C₅₋₁₀aryl. Non-limiting examples of aryl comprise phenyl, biphenylyl, biphenylenyl, or 1- or 2-naphthanelyl; 1-, 2-, 3-, 4-, 5- or 6-tetralinyl (also known as “1,2,3,4-tetrahydronaphtalene); 1-, 2-, 3-, 4-, 5-, 6-, 7- or 8-azulenyl, 4-, 5-, 6 or 7-indenyl; 4- or 5-indanyl; 5-, 6-, 7- or 8-tetrahydronaphthyl; 1,2,3,4-tetrahydronaphthyl; and 1,4-dihydronaphthyl; 1-, 2-, 3-, 4- or 5-pyrenyl. When the suffix “ene” is used in conjunction with an aryl group, this is intended to mean the aryl group as defined herein having two single bonds as points of attachment to other groups.

The term “alkoxy” or “alkyloxy” or “alkoxide”, as a group or part of a group, refers to a group having the Formula —OR^{x1} wherein R^{x1} is alkyl as defined herein above. Examples of suitable alkyloxy include C₁₋₂₀alkyloxy, or C₁₋₁₈alkyloxy, or C₁₋₁₂alkyloxy, or C₁₋₆alkyloxy. Non-limiting examples of suitable alkoxy include, but are not limited to methoxy, ethoxy, propoxy, isopropoxy, butoxy, isobutoxy, sec-butoxy, tert-butoxy, pentyloxy and hexyloxy.

The term “aryloxy” or “aryloxide”, as a group or part of a group, refers to a group having the formula —OR^{x2} wherein R^{x2} is aryl as defined herein above. Examples of suitable aryloxy include C₅₋₃₀aryloxy, or C₆₋₃₀aryloxy, or C₆₋₁₂aryloxy.

The term “hydroxyl” or “hydroxy”, as a group or part of a group, refers to the group-OH. The term “amino” refers to the group —NH₂.

Preferred embodiments of this invention are set herein below. Each embodiment of the invention so defined may be combined with any other embodiment unless clearly indicated to the contrary. In particular, any feature indicated as being preferred or advantageous may be combined with any other feature or features indicated as being preferred or advantageous.

According to a first aspect, the present invention relates to a process for dimerising a C₅₋₂₀ alcohol to a C₁₀₋₄₀ alcohol. The process preferably comprises the steps of:
contacting the C₅₋₂₀ alcohol with a catalytic mixture; and,
dimerising the C₅₋₂₀ alcohol in the catalytic mixture to form a C₁₀₋₄₀ alcohol.

The catalytic mixture preferably comprises:
a base;
a catalyst comprising a (L)ₙM(OH)ₙ(H₂O)ₘ type complex;
   wherein M is a metal selected from the group comprising: Fe, Ru, Os, Zn, Ni;
   and, wherein L is a ligand;
a Zn additive; and,
a solvent.

The dimerisation preferably occurs in the sense of a Guerbet reaction. In a Guerbet reaction, a single starting alcohol can be used; however, also several different alcohols may be used as starting alcohols. The main product of the Guerbet reaction are dimerisation products. However, in minor amounts also higher homologs may be present, such trimerisation, resulting from the fact that the primary dimerisation with unreacted starting alcohols also react in the manner of a Guerbet reaction.

Therefore, when referring to a “process for the dimerisation of alcohols in the sense of a Guerbet reaction”, this includes not only the formation of dimerisation products but also the formation of higher homologs. If one uses exclusively a primary or secondary monomeric alcohol, then this is referred to as a “classical” Guerbet reaction. If one uses several primary and/or secondary monomeric alcohols, then this is referred to as a “mixed” Guerbet reaction.

In some preferred embodiments, the C₅₋₂₀ alcohol is a C₅ alcohol. In some embodiments, the C₅₋₂₀ alcohol is a fusel alcohol, for example selected from the group comprising: amyl alcohol (2-methyl-1-butanol), isoamyl alcohol (3-methyl-1-butanol), iso-butyl alcohol, 1-propanol, n-amyl alcohol, n-butyl alcohol, and methionol; preferably selected from the group comprising: amyl alcohol (2-methyl-1-butanol), isoamyl alcohol (3-methyl-1-butanol), iso-butyl alcohol, 1-propanol. Most preferably, the C₅₋₂₀ alcohol is isoamyl alcohol. The invention, however, is not limited to the use of just isoamyl alcohol. Other isomeric C₅ alcohols or C₆, C₇, C₈ up to C₂₀ alcohols may also be used. In some preferred embodiments, the C₅₋₂₀ alcohol is a C₅ alcohol obtained from a side stream of bio-ethanol production, or the product of a hydroformylation (oxo-process) and subsequent hydrogenation of the corresponding olefin compound. If the alcohol is derived from bio-ethanol production, it would qualify as green.

Isoamyl alcohol has the formula (H₃C—)₂CH—CH₂—CH₂—OH, and may also be referred to as 3-methyl-butan-1-ol, isopentyl alcohol, or isopentanol. Isoamyl alcohol is an ingredient in the production of banana oil, an ester found in nature and also produced as a flavouring in industry. It is a common fusel alcohol, produced as a major by-product of ethanol fermentation. In some preferred embodiments, isoamyl alcohol is obtained from a side stream of bio-ethanol production or the product of a hydroformylation (oxo-process) and subsequent hydrogenation of the corresponding isobutene compound. If the isoamyl alcohol is derived from bio-ethanol production, it would qualify as green.

In some preferred embodiments, the C₁₀₋₄₀ alcohol is a C₁₀ alcohol. Most preferably, the C₁₀₋₄₀ alcohol is 2-isopropyl-5-methyl-1-hexanol, or any structural isomer known to those skilled in the art.

The catalytic mixture preferably comprises a catalyst. The preferred amount of catalyst used may depend to some extent upon the use of the C₅₋₂₀ alcohol (preferably isoamyl alcohol) neat or with an additional solvent so as to stay within the confines of the operating temperature, pressure, time, and other variables known to those skilled in the art. Preferably, the amount of the catalyst used is between 0.0001-20.0%, more preferably 0.01-10.0% and most preferably 1.00-5.0% by weight based on the weight of the C₅₋₂₀ alcohol (preferably isoamyl alcohol).

The catalyst preferably comprises an (L)ₙM(OH)ₙ(H₂O)ₘ type complex. M is a metal, preferably selected from the group comprising: Fe, Ru, Os, Zn, Ni. In some preferred embodiments, the metal M is Fe or Ru, preferably Fe. In some preferred embodiments, the catalyst comprises an iron dichloride hydrate or solvate salt or ruthenium trichloride hydrate or solvate salt.

The person skilled in the art understands that the catalyst preferably comprising an (L)ₙM(OH)ₙ(H₂O)ₘ type complex refers to a “complex” such that the indices “n” and “m” cannot simultaneously be 0. Therefore, the catalyst comprises an (L)ₙM(OH)ₙ(H₂O)ₘ type complex comprising at least one ligand L.

The preferred amount of the ligand used may depend to some extent upon the use of catalyst. Preferably, the amount of the ligand used is between 0.1-200 molar equivalents based on the molecular weight of the reaction mixture.

In some preferred embodiments, the ligand L is selected from the group comprising: a halide, a hydride, an alkoxide, an aryloxide, an amide, an acetate, an acetylacetonate, an alkyl, an aryl, CO, NO, phosphine, pyridine, an alkene, an alkyne, N-heterocyclic carbene, cyclopentadiene, a mono-dentate phosphine of formula PR¹R²R³, a bidentate phosphine such as R¹R²P—PR¹R², a methylene linked R¹R²P—CH₂—PR¹R², R¹R²P—(CH₂)ₙ—PR¹R², or R¹R²P—(CH₂)ₙ—NR⁴—(CH₂)ₙ—PR¹R²;
wherein each R¹, R², R³ and/or R⁴ is independently selected from alkyl or aryl, with optional variable functionality such as sulfonate, halide, and wherein R¹, R², R³, and R⁴ can be identical or different; preferably wherein R¹, R², R³, and R⁴ are all phenyl.

In some preferred embodiments, the ligand L is selected from the group comprising: a mono-dentate phosphine of formula PR¹R²R³, a bidentate phosphine such as R¹R²P—PR¹R², a methylene linked R¹R²P—CH₂—PR¹R², R¹R²P—(CH₂)ₙ—PR¹R², or R¹R²P—(CH₂)ₙ—NR⁴—(CH₂)ₙ—PR¹R²;
wherein each R¹, R², R³ and/or R⁴ is independently selected from alkyl or aryl, with optional variable functionality such as sulfonate, halide, and wherein R¹, R², R³, and R⁴ can be identical or different; preferably wherein R¹, R², R³, and R⁴ are all phenyl.

Phosphines were found to be relatively stable under basic conditions tested as opposed to phosphites, even at temperatures of 220° C., is illustrated in FIG. 3. In some embodiments, the ligand is a chelating ligand, preferably a bidentate ligand.

The catalytic mixture preferably comprises a Zn additive. Zinc metal or Zn (0) by itself was previously known to be inactive for Guerbet reactions under the conditions listed for isoamyl alcohol. Unexpectedly, the present inventors found the Zn to enhance the conversion.

The amount of Zn metal additive used preferably comprises from at least 1 to at most 500 equivalents compared to the metal of the catalyst (preferably Fe), more preferably from at least 2 to at most 100 equivalents compared to the metal of the catalyst (preferably Fe), and most preferably from at least 2 to at most 50 equivalents compared to the metal of the catalyst (preferably Fe). The Zn additive is preferably added as a powder, preferably a fine powder. In some embodiments, Zn metal shavings may be added. Preferably powder is added, since it affects diffusion/mixing of the reaction mixture to a lesser extent.

It should be noted that in the process as disclosed herein, the Zn additive refers to a separate component in the catalyst mixture. In particular, the Zn additive is a component different from the base and the catalyst referred to herein. The term “additive” as used herein is a substance or compound in the catalyst mixture that can be configured to modify or enhance the reactivity and/or selectivity of a chemical reaction.

The catalytic mixture preferably comprises a base. The alkali base may be used in a catalytic amount. Preferably, the amount of the base used is from at least 1.0 to at most 30.0%, more preferably from at least 2.0 to at most 20.0%, and most preferably from at least 5.0 to at most 10.0% by weight based on the weight of the C₅₋₂₀ alcohol (preferably isoamyl alcohol).

In some preferred embodiments, the base is selected from the group comprising: potassium hydroxide, sodium hydroxide, potassium isoamylate, sodium isoamylate, potassium t-butoxide, sodium t-butoxide, or combinations thereof, for example a eutectic mixture of sodium and potassium hydroxide. Alkoxides of other branched alcohols may also be used as a base.

In some preferred embodiments, the basic aqueous solvent phase comprises an alkali hydroxide. In some preferred embodiments, the base comprises potassium hydroxide. KOH is widely available, and inexpensive to store, and handle compared to sodium metal.

Sodium with isoamyl alcohol gives sodium amylate which is prepared in situ. It was expected that amylate would provide better conversions over KOH, as amylate is more soluble in isoamyl alcohol making it more homogeneous as KOH takes longer to dissolve. Unexpectedly, in Comparative Example 7 it was found to result in lower conversions than KOH. Sodium amylate should also react with the water formed during the conversion and give NaOH and isoamyl alcohol i.e., the effect of water formed should be lower on conversion. However, this was not found to be the case.

The catalytic mixture preferably comprises a solvent. In order to stay below the operating conditions of temperatures less than 200° C. and pressure of less than 20 bar, one may advantageously opt to use an additional solvent. The amount of solvent may vary from 0.5-200, 0.5-100, preferably 0.5-50, more preferably 0.5-10 and most preferably 0.5-5 by volume with respect to the feed C₅₋₂₀ alcohol (preferably isoamyl alcohol) used.

In some preferred embodiments, the solvent is selected from the group comprising: poly(ethylene glycol) (PEG) monoalkyl ethers, aliphatics, aromatics, aromatics with aliphatic substitutions, xylenes, cycloalkanes, substituted cycloalkanes, naphthenes, indenes, fluorene, biphenyls, Petrosolv 200-300, Petroflux ND, Petrosolv 250-450, SOLGAD 150, SOLGAD 200, SOLGAD 200 ULN, SOLGAD 150 ULN, sulfolane, dimethyl sulfoxide, dimethyl formamide, N-methyl pyrrolidone, N,N-dimethyl acetamide, 1,4-dioxane, anisole, propylene carbonate, benzyl alcohol, N-methylpyrrolidone, N-ethylpyrrolidone, N-cyclohexylpyrrolidone, N-octylpyrrolidone, ethyllactate, butyllactate, morpholine, glycerin, glycerin-mono-tert-butyl-ether, glycerin-di-tert-butyl-ether, glycerin-tri-tert-butyl-ether, acetonitrile, propionitrile, di-arylether, alkyl aryl ethers, ionic liquids, dicarboxylic/tricarboxylic ester-based plasticisers, bis(2-ethylhexyl) phthalate, diisononyl phthalate, bis(n-butyl) phthalate, butyl benzyl phthalate, diisodecyl phthalate, di-n-octyl phthalate, diisooctyl phthalate, diethyl phthalate, diisobutyl phthalate, di-n-hexyl phthalate, trimellitates, trimethyl trimellitate, tri-(2-ethylhexyl) trimellitate, tri-(n-octyl,n-decyl) trimellitate, tri-(heptyl, nonyl) trimellitate, n-octyl trimellitate, bis(2-ethylhexyl) adipate, dimethyl adipate, monomethyl adipate, dioctyl adipate, sebacate-based plasticisers, dibutyl sebacate, maleates, dibutyl maleate, diisobutyl maleate, benzoates, epoxidised vegetable oils, sulfonamides, N-ethyl toluene sulfonamide (ortho and para isomers), N-(2-hydroxypropyl)benzene sulfonamide, N-(n-butyl)benzene sulfonamide, organophosphates, tricresyl phosphate, tributyl phosphate, glycols, polyethers, triethylene glycol dihexanoate, tetraethylene glycol diheptanoate, polybutene, acetylated monoglycerides, alkyl citrates, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trihexyl o-butyryl citrate, trimethyl citrate, alkyl sulphonic acid phenyl ester, vinyl chloride copolymers, 1,2-cyclohexane dicarboxylic acid diisononyl ester, biphenyl, triphenylmethane, polyaromatic hydrocarbons, tetralin, naphthalene, tetrahydro anthracene, anthracene, hexahydro pyrene, trihydropyrene, pyrene, phenanthrene, chrysene, paraffins, paraffinic intermediates, light naphtha, mid-naphtha, light gas oil, mid gas oil, heavy gas oil, light vacuum gas oil, mid-vacuum gas oil, heavy vacuum gas oil, vacuum residue, linear alkyl benzenes, vegetable oils, fatty acid methyl esters, animal fats/tallow, optionally substituted derivatives thereof, and combinations thereof.

In some preferred embodiments, the solvent comprises a compound selected from the group comprising: xylenes, mesitylene, biphenyls, and Petrosolv 200-300; preferably xylenes. Xylenes are preferred due to their availability and commercial viability. The inventors surprisingly found that the solvent did not dilute and lower conversion, but provided higher conversion instead.

The optimum temperature employed in the process of the invention may vary with the alcohol to catalyst ratio, and desired process pressure. Generally, the present process can be carried out at the reflux temperature of isoamyl alcohol. While a temperature of as low as 120° C. can be used and satisfactory results are obtained, it is preferable to employ a temperature of at least 160° C. but less than 200° C. In some preferred embodiments, the step of dimerising the C₅₋₂₀ alcohol in the catalytic mixture to form a C₁₀₋₄₀ alcohol, is performed at a temperature of at least 120° C. and at most 200° C., for example at least 160° C. to at most 200° C.

In some preferred embodiments, the step of dimerising the C₅₋₂₀ alcohol in the catalytic mixture to form a C₁₀₋₄₀ alcohol, is performed at a pressure of at least 0.01 bar and at most 10 bar.

The specific time period over which the conversion may be achieved upon reaching a target temperature and pressure, i.e., the “retention time or residence time” may depend on a number different factors including, for example, the type of solvent used, the amount of C₅₋₂₀ alcohol (preferably isoamyl alcohol), the types of catalyst/s as defined herein, in the mixture and their various concentration/s, and/or the type of reactor apparatus as described herein in which the methods are performed. These and other factors may be varied to optimise a given method to maximise the yield and/or reduce the processing time. Preferably, the retention time is sufficient to convert all or substantially all the C₅₋₂₀ alcohol (preferably isoamyl alcohol) used as a feed into the product C₁₀₋₄₀ alcohol. The time required for the reaction is not particularly critical and can be varied as desired although it should be sufficiently long to ensure obtaining an appreciable conversion of the C₅₋₂₀ alcohol (preferably isoamyl alcohol) to the product C₁₀₋₄₀ alcohol, but not so long as to allow further condensation to form trimers and tetramers. In general, a reaction time of 5 minutes to 24 h or more is adequate although best results are obtained with a period of about 0.5-6 h at optimum temperature, pressure, and catalyst concentration. In some preferred embodiments, the step of dimerising the C₅₋₂₀ alcohol in the catalytic mixture to form a C₁₀₋₄₀ alcohol, is performed during a residence time of at least 30 minutes to at most 6 hours.

A commercial process to convert isoamyl alcohol to 2-isopropyl-5-methyl-1-hexanol using aspects of the present invention may be realized in many different ways, for example depending on the preferred conditions for the alcohol to be converted. By way of example, an industrial process employing features of the present invention may include some or all of the following steps.

The isoamyl alcohol to be converted may be pre-treated to achieve the preferred conditions for reaction coming from the fermentation plant, which will vary according to the feedstock that is used to produce ethanol. Pre-treatment may include the removal of trace contaminants deleterious to the process, and/or heating/cooling to preferred reaction conditions (phase, temperature, and pressure), and/or distillation. The catalyst may be prepared when required either offsite by specialist suppliers or onsite, depending on the type of reaction system and the ease of catalyst preparation, transportation, and storage.

Following the pre-treatment, a mixture of feed alcohol and catalyst solution may be fed to the system. Separate pumps may be used to dose alcohol and aa catalyst solution. Both streams may flow to a mixer. A reactor system, which may comprise a heating thermostat and temperature control, may be used to pre-heat the mixture, for example to 50° C.

The reactor preferably allows for control over residence time, mixing rate, temperature ramping rate as well as final reaction temperature, for example of 180-200° C. The processes of heating/pressurisation and cooling/de-pressurisation may be facilitated by performing the methods of the invention in a continuous flow system which may be a CSTR or tubular reactor or a microchannel reactor. The process steps involved such as heating/pressurization and cooling/de-pressurization may be facilitated by performing the methods of the invention in a continuous flow system which may be a CSTR or tubular reactor or a microchannel reactor apparatus. In some preferred embodiments, the step of dimerising the C₅₋₂₀ alcohol in the catalytic mixture to form a C₁₀₋₄₀ alcohol, is performed in a continuous flow system, preferably selected from the group comprising: a CSTR, a tubular reactor, or a microchannel reactor apparatus.

An optimum reaction temperature for the production of Guerbet alcohols is typically in the range of 100-180° C. and pressure of less than 20 bar with a residence time of less than 10 hours. Lowering the reaction temperature as a result also results in lower pressures which enable the technical realization of short-chain Guerbet alcohols in customary industrial plants (p max=12 bar). It is preferable to operate at pressures of from 0.01 to 10 bar and in particular from 0.1 to 8 bar.

The reaction pressure (for example 8 to 10 bar) may be controlled by pressure regulator. The pressure elevation may be realised by a feed pump (A), and/or one or more catalyst solution pumps.

Once the mixture has fully reacted, the products are preferably separated from the catalyst solution in a separator, for example by means of pressure, temperature, and/or velocity control. In some embodiments, the catalyst solution is recycled through a pump, and directed back to a mixer.

The alcohol products that are separated in a separator, preferably flow towards a distillation system. A cooling thermostat may be used to perform a fractionated distillation. The quantity of fractionated product streams can be modified in regard of the process conditions and requirements.

According to a second aspect, the present invention relates to use of a catalyst as described herein, in the dimerisation of a C₅₋₂₀ alcohol to a C₁₀₋₄₀ alcohol.

The independent and dependent claims set out particular and preferred features of the invention. Features from the dependent claims may be combined with features of the independent or other dependent claims as appropriate.

### EXAMPLES

### Materials

Commercially obtained reagents were used as received from appropriate commercial vendors, such as VWR or Merck, without any further purification. Isoamyl alcohol (technical grade)—alternatively crude fusel alcohol resulting from a bio-ethanol production facility may also be used—zinc fine powder, and technical grade xylenes mixture, were purchased from VWR Chemicals. KOH (>85%) was purchased from Carl Roth GmbH. Iron (II) chloride tetrahydrate was purchased from Merck. Triphenyl phosphine was purchased from Acros Organics. Celite 535 was purchased from Macherey-Nagel GmbH & Co. KG.

### Methods

All reactions were conducted with a 2 L Parr stainless steel reactor. All test runs were prepared in air unless stated otherwise. All components were found to be stable in air.

### Analytics

Gas chromatogram-flame ionization detector (GC-FID), gas chromatogram-mass spectra (GC-MS) analyses were carried out on an Agilent 8890 using a HP-5 ms column, in-house. All crude solutions were prepared in diethyl ether in 2 ml vials with PTFE screw caps.

TABLE 1
Chromatographic Conditions for raw
oligomerization product analysis

| | |
|---|---|
| GC | Agilent 8890/5977B GC/MSD |
| Sampler | Agilent 7650A, 5.0-μL syringe |
| Carrier | Hydrogen 30 cm/s, constant flow |
| Inlet | Split (15:1); 250° C., purge flow 22.5 mL/min |
| Inlet liner | Deactivated dual taper direct connect |
| Column | Agilent HP-5ms 30 m × 0.25 mm × 0.25 μm |
| Oven | 42° C. (6 min) to 220° C. (10° C./min), |
| | 15° C./min to 310° C. |
| Detection | MSD source at 230° C., quadrupole at 150° C., |
| | scan range 30 to 300 amu |

### Comparative Example 1

Isoamyl alcohol, iron as a dichloride·tetrahydrate salt, and KOH in molar ratio (73.1:1.0:21.3), were premixed at ambient temperature in air, then heated at 40° C. (5 h), then ramped at 20° C. increments to 180° C. in 1 h, time at temperature T=7 h. Upon completion of the reaction, the reactor was cooled to ambient temperature and pressure released. The reaction mixture was filtered through celite and analysed with GC-FID and GC-MS. The selectivity was determined for example by integration of GC signals for 2-isopropyl-5-methyl-1-hexanol compared with all the other components in the product other than isoamyl alcohol. GC-trace showed trace levels of conversion.

### Comparative Example 2

Isoamyl alcohol, iron as a dichloride·tetrahydrate salt, PPh₃, KOH in molar ratio (73.1:1.0:2.0:21.3), were premixed at ambient temperature in air, then heated at 40° C. (5 h), then ramped at 20° C. increments to 180° C. in 1 h, time at temperature T=7 h. Upon completion of the reaction, the reactor was cooled to ambient temperature and pressure released. The reaction mixture was filtered through celite and analysed with GC-FID and GC-MS. The selectivity was determined for example by integration of GC signals for 2-isopropyl-5-methyl-1-hexanol compared with all the other components in the product other than isoamyl alcohol. GC-trace showed 40.1% conversion, >99% selectivity.

### Comparative Example 3

Isoamyl alcohol, iron as a dichloride·tetrahydrate salt, PPh₃, KOH in molar ratio (73.1:1.0:2.0:21.3), and xylenes (isoamyl alcohol:xylenes; 1:1 v/v) were premixed at ambient temperature in air, then heated at 40° C. (5 h), then ramped at 20° C. increments to 180° C. in 1 h, time at temperature T=7 h. Upon completion of the reaction, the reactor was cooled to ambient temperature and pressure released. The reaction mixture was filtered through celite and analysed with GC-FID and GC-MS. The selectivity was determined for example by integration of GC signals for 2-isopropyl-5-methyl-1-hexanol compared with all the other components in the product other than isoamyl alcohol. GC-trace showed 53.3% conversion, >99% selectivity.

### Comparative Example 4

Isoamyl alcohol, iron as a dichloride·tetrahydrate salt, PPh₃, KOH, zinc in molar ratio (73.1:1.0:2.0:21.3:6.1), were premixed at ambient temperature in air, then heated at 40° C. (5 h), then ramped at 20° C. increments to 180° C. in 1 h, time at temperature T=7 h. Upon completion of the reaction, the reactor was cooled to ambient temperature and pressure released. The reaction mixture was filtered through celite and analysed with GC-FID and GC-MS. The selectivity was determined for example by integration of GC signals for 2-isopropyl-5-methyl-1-hexanol compared with all the other components in the product other than isoamyl alcohol. GC-trace showed 49.7% conversion, >99% selectivity.

### Example 5

Isoamyl alcohol, iron as a dichloride·tetrahydrate salt, PPh₃, KOH, zinc in molar ratio (73.1:1.0:2.0:21.3:6.1), and xylenes (isoamyl alcohol:xylenes; 1:1 v/v) were premixed at ambient temperature in air, then heated at 40° C. (5 h), then ramped at 20° C. increments to 180° C. in 1 h, time at temp T=7 h. Upon completion of the reaction, the reactor was cooled to ambient temperature and pressure released. The reaction mixture was filtered through celite and analysed with GC-FID and GC-MS. The selectivity is determined for example by integration of GC signals for 2-isopropyl-5-methyl-1-hexanol compared with all the other components in the product other than isoamyl alcohol. GC-trace showed 58.5% conversion, >99% selectivity.

### Example 6

Isoamyl alcohol, iron as a dichloride·tetrahydrate salt, PPh₃, KOH, zinc in molar ratio (73.1:1.0:2.0:21.3:6.1), and xylenes (isoamyl alcohol:xylenes; 1:2 v/v) were premixed at ambient temperature in air, then heated at 40° C. (5 h), then ramped at 20° C. increments to 180° C. in 1 h, time at temp T=7 h. Upon completion of the reaction, the reactor was cooled to ambient temperature and pressure released. The reaction mixture was filtered through celite and analysed with GC-FID and GC-MS. The selectivity was determined for example by integration of GC signals for 2-isopropyl-5-methyl-1-hexanol compared with all the other components in the product other than isoamyl alcohol. GC-trace showed 64.4% conversion, >99% selectivity.

### Comparative Example 7

Isoamyl alcohol, iron as a dichloride·tetrahydrate salt, PPh₃, Na in molar ratio (73.1:1.0:2.0:17.3), were premixed at ambient temperature in air, then heated at 40° C. (5 h), then ramped at 20° C. increments to 180° C. in 1 h, time at temperature T=7 h. Upon completion of the reaction, the reactor was cooled to ambient temperature and pressure released. The reaction mixture was filtered through celite and analysed with GC-FID and GC-MS. The selectivity is determined for example by integration of GC signals for 2-isopropyl-5-methyl-1-hexanol compared with all the other components in the product other than isoamyl alcohol. GC-trace showed 32.8% conversion, >99% selectivity.

### Example 8—Temperature Versus Pressure Measurement

Isoamyl alcohol, iron as a dichloride·tetrahydrate salt or ruthenium trichloride·trihydrate, PPh₃, KOH, zinc in molar ratio (73.1:1.0:2.0:21.3:6.1), and xylenes (isoamyl alcohol:xylenes; 1:2 v/v) were premixed at ambient temperature in air, then heated at 40° C. (5 h), then ramped at 10° C. increments to 230° C. at each 10° C. increments the temperature and pressure were measured as demonstrated in FIG. 3. Upon completion of the reaction, the reactor was cooled to ambient temperature and pressure released. Pressure generated with ruthenium was far more than iron as shown.

### Example 9—Distillation

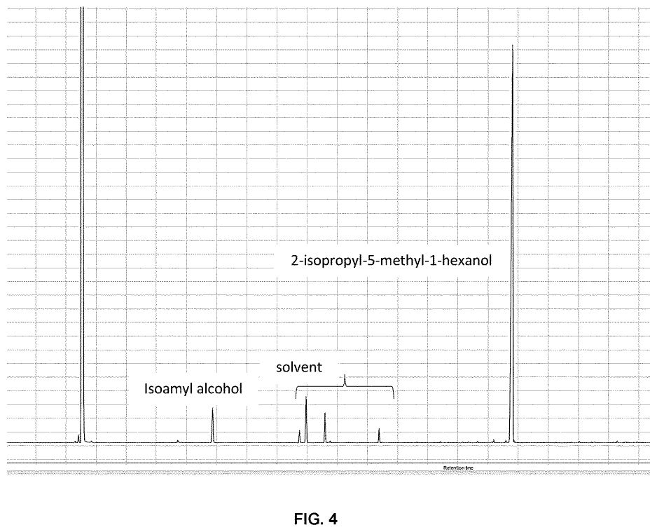

A representative crude sample reaction mixture (1542 g) resulting from the tests with a GC-FID such as the one shown in FIG. 4 was transferred to a 3000 mL round bottom flask equipped with a stir bar and a fractionating column 60 cm long fitted with iron or copper wool to increase the number of theoretical plates. The distillation is preferably carried out at atmospheric pressure, although it is possible to operate at sub-atmospheric or super-atmospheric pressures, if desirable under certain circumstances. In general, the number of trays in the column and amount of heat transferred to the material being purified in the column are sufficient to produce a liquid stream of purified 2-isopropyl-5-methyl-1-hexanol containing at least about 99% of 2-isopropyl-5-methyl-1-hexanol as judged by gas chromatographic analysis.

The distillation products have the following compositions as judged by GC-FID:
Fraction 1:131-140° C., 1250 g (mixture of isoamyl alcohol and xylenes solvent);
Fraction 2:111.5° C. at 30 mmHg (214° C. at 760 mmHg) 241 g (2-isopropyl-5-methyl-1-hexanol);
Fraction 3: Bottoms (residue) rest.

This example demonstrates that the inventors successfully distilled the sample.

### Example 10—Industrial Production Process

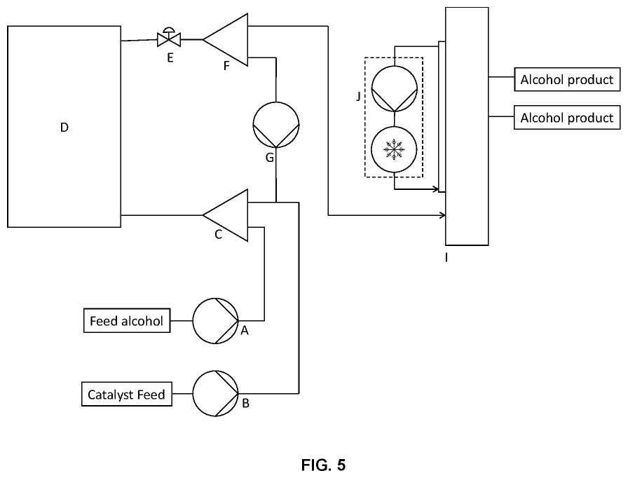

FIG. 5 illustrates commercial process to convert isoamyl alcohol to 2-isopropyl-5-methyl-1-hexanol using aspects of the present invention.

Following the pre-treatment, a mixture of feed alcohol and catalyst solution are fed to the system. Pump (A) is dosing alcohol, pump (B) is dosing a catalyst solution. Both streams flow to mixer (C). A reactor system (D), consists of a heating thermostat and temperature control allows for pre heating the mixture to 50° C. The reactor allows for control over residence time, mixing rate, temperature ramping rate as well as final reaction temperature of 180-200° C. The reactor may be a continuous flow system which may be a CSTR or tubular reactor or a microchannel reactor.

The reaction pressure (8 to 10 bar) is controlled by pressure regulator (E). The pressure elevation is realized by feed pump (A), catalyst solution pump (B) and/or catalyst solution pump (G).

Once the mixture has fully reacted, the products are separated from the catalyst solution in separator (F) by means of pressure, temperature, and velocity control. The catalyst solution is recycled through pump (F) and directed back to mixer (C).

The alcohol products that are separated in separator (F), flow towards a distillation system (I) where cooling thermostat (J) is used to perform a fractionated distillation. The quantity of fractionated product streams can be modified in regard of the process conditions and requirements.

1. A process for dimerising a C₅₋₂₀ alcohol to a C₁₀₋₄₀ alcohol, the process comprising the steps of:
contacting the C₅₋₂₀ alcohol with a catalytic mixture comprising:
a base;
a catalyst comprising a (L)ₙM(OH)ₙ(H₂O)ₘ type complex, comprising at least one ligand L;
wherein M is a metal selected from Fe or Ru; and,
wherein L is a phosphine ligand;
a Zn additive; and,
a solvent; and,
dimerising the C₅₋₂₀ alcohol in the catalytic mixture to form a C₁₀₋₄₀ alcohol.

2. The process according to claim 1, wherein the C₅₋₂₀ alcohol is a C₅ alcohol.

3. The process according to claim 1, wherein the C₁₀₋₄₀ alcohol is a C₁₀ alcohol.

4. The process according to claim 1, wherein the metal M is Fe.

5. The process according to claim 1, wherein the catalyst comprises an Fe dichloride hydrate salt.

6. (canceled)

7. The process according to claim 1, wherein the ligand L is selected from the group comprising: a mono-dentate phosphine of formula PR¹R²R³, a bidentate phosphine such as R¹R²P—PR¹R², a methylene linked R¹R²P—CH₂—PR¹R², R¹R²P—(CH₂)ₙ—PR¹R², or R¹R²P—(CH₂)ₙ—NR⁴—(CH₂)ₙ—PR¹R²; wherein each R¹, R², and/or R³ is independently selected from alkyl or aryl, with optional variable functionality such as sulfonate, halide, and wherein R¹, R², R³, R⁴ can be identical or different.

8. The process according to claim 1, wherein the base is selected from the group comprising: potassium hydroxide, sodium hydroxide, potassium isoamylate, sodium isoamylate, potassium t-butoxide, sodium t-butoxide, or combinations thereof.

9. The process according to claim 1, wherein the base comprises potassium hydroxide.

10. The process according to claim 1, wherein the solvent is selected from the group comprising: poly(ethylene glycol) (PEG) monoalkyl ethers, aliphatics, aromatics, aromatics with aliphatic substitutions, xylenes, cycloalkanes, substituted cycloalkanes, naphthenes, indenes, fluorene, biphenyls, Petrosolv 200-300, Petroflux ND, Petrosolv 250-450, SOLGAD 150, SOLGAD 200, SOLGAD 200 ULN, SOLGAD 150 ULN, sulfolane, dimethyl sulfoxide, dimethyl formamide, N-methyl pyrrolidone, N,N-dimethyl acetamide, 1,4-dioxane, anisole, propylene carbonate, benzyl alcohol, N-methylpyrrolidone, N-ethylpyrrolidone, N-cyclohexylpyrrolidone, N-octylpyrrolidone, ethyllactate, butyllactate, morpholine, glycerin, glycerin-mono-tert-butyl-ether, glycerin-di-tert-butyl-ether, glycerin-tri-tert-butyl-ether, acetonitrile, propionitrile, di-arylether, alkyl aryl ethers, ionic liquids, dicarboxylic/tricarboxylic ester-based plasticisers, bis(2-ethylhexyl) phthalate, diisononyl phthalate, bis(n-butyl) phthalate, butyl benzyl phthalate, diisodecyl phthalate, di-n-octyl phthalate, diisooctyl phthalate, diethyl phthalate, diisobutyl phthalate, di-n-hexyl phthalate, trimellitates, trimethyl trimellitate, tri-(2-ethylhexyl) trimellitate, tri-(n-octyl,n-decyl) trimellitate, tri-(heptyl, nonyl) trimellitate, n-octyl trimellitate, bis(2-ethylhexyl) adipate, dimethyl adipate, monomethyl adipate, dioctyl adipate, sebacate-based plasticisers, dibutyl sebacate, maleates, dibutyl maleate, diisobutyl maleate, benzoates, epoxidised vegetable oils, sulfonamides, N-ethyl toluene sulfonamide (ortho and para isomers), N-(2-hydroxypropyl) benzene sulfonamide, N-(n-butyl) benzene sulfonamide, organophosphates, tricresyl phosphate, tributyl phosphate, glycols, polyethers, triethylene glycol dihexanoate, tetraethylene glycol diheptanoate, polybutene, acetylated monoglycerides, alkyl citrates, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trihexyl o-butyryl citrate, trimethyl citrate, alkyl sulphonic acid phenyl ester, vinyl chloride copolymers, 1,2-cyclohexane dicarboxylic acid diisononyl ester, biphenyl, triphenylmethane, polyaromatic hydrocarbons, tetralin, naphthalene, tetrahydro anthracene, anthracene, hexahydro pyrene, trihydropyrene, pyrene, phenanthrene, chrysene, paraffins, paraffinic intermediates, light naphtha, mid-naphtha, light gas oil, mid gas oil, heavy gas oil, light vacuum gas oil, mid-vacuum gas oil, heavy vacuum gas oil, vacuum residue, linear alkyl benzenes, vegetable oils, fatty acid methyl esters, animal fats/tallow, optionally substituted derivatives thereof, and combinations thereof.

11. The process according to claim 1, wherein the solvent comprises xylenes.

12. The process according to claim 1, wherein the step of dimerising the C₅₋₂₀ alcohol in the catalytic mixture to form a C₁₀₋₄₀ alcohol, is performed at a temperature of at least 120° C. and at most 200° C.

13. The process according to claim 1, wherein the step of dimerising the C₅₋₂₀ alcohol in the catalytic mixture to form a C₁₀₋₄₀ alcohol, is performed at a pressure of at least 0.01 bar and at most 10 bar.

14. The process according to claim 1, wherein the step of dimerising the C₅₋₂₀ alcohol in the catalytic mixture to form a C₁₀₋₄₀ alcohol, is performed during a residence time of at least 30 minutes to at most 6 hours.

15. The process according to claim 1, wherein the step of dimerising the C₅₋₂₀ alcohol in the catalytic mixture to form a C₁₀₋₄₀ alcohol, is performed in a continuous flow system.

16. The process according to claim 1, wherein the solvent is selected from the group comprising: xylenes, mesitylene, biphenyls, Petrosolv 200-300, and combinations thereof.

17. The process according to claim 1, wherein the step of dimerising the C₅₋₂₀ alcohol in the catalytic mixture to form a C₁₀₋₄₀ alcohol, is performed in a continuous flow system selected from the group comprising: a CSTR, a tubular reactor, or a microchannel reactor apparatus.

18. The process according to claim 1, wherein the ligand L is selected from the group comprising: a mono-dentate phosphine of formula PR¹R²R³, a bidentate phosphine such as R¹R²P—PR¹R², a methylene linked R¹R²P—CH₂—PR¹R², R¹R²P—(CH₂)ₙ—PR¹R², or R¹R²P—(CH₂)ₙ—NR⁴—(CH₂)ₙ—PR¹R²; wherein each R¹, R², and/or R³ is independently selected from alkyl or aryl, with optional variable functionality such as sulfonate, halide, and wherein R¹, R², R³, R⁴ are all phenyl.

19. The process according to claim 1, wherein the C₅₋₂₀ alcohol is isoamyl alcohol.

20. The process according to claim 1, wherein the C₁₀₋₄₀ alcohol is 2-isopropyl-5-methyl-1-hexanol.
